(19) United States
(12) Patent Application Publication
(10) Pub. No.: US 2011/0021904 A1
(43) Pub. Date: Jan. 27, 2011

(54) DYNAMIC PLANNING TOOL FOR USE IN CONTRAST-ENHANCED DYNAMIC SCAN IN MAGNETIC RESONANCE IMAGING

(75) Inventor: Johannes Burrman, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/933,115

(22) PCT Filed: Mar. 11, 2009
(86) PCT No.: PCT/IB09/51008

§ 371 Date: Sep. 17, 2010

(30) Foreign Application Priority Data
Mar. 18, 2008 (EP) 08152868.9

(51) Int. Cl. A61B 5/055

(52) U.S. Cl. 600/420

(57) ABSTRACT

The timing scheme of a dynamic contrast-enhanced MRI scan of the breast is critical for any subsequent analysis or CAD. This invention proposes a planning device configured to calculate the timing scheme of a dynamic MRI investigation, to be integrated in either the MRI scanner or a breast analysis or CAD software package. The planned scan may be transferred to the scanner by hand or by means of an ExamCard. The planning may also be used as the input for an analysis or CAD software pack-age.

### FIELD OF THE INVENTION

This invention pertains in general to the field of Medical Imaging. More particularly the invention relates to a dynamic planning tool for use in contrast-enhanced dynamic scan in magnetic resonance imaging.

### BACKGROUND OF THE INVENTION

Magnetic Resonance Imaging (MRI) examinations for breast cancer include a dynamic contrast-enhanced scan, wherein the intensity in each voxel of the acquired image as a function of time is indicative of the underlying pathology.

During the image acquisition scan, a dynamic pre-contrast scan is performed, and subsequently contrast agent is injected intravenously. There are several contrast agents available, e.g. water, taken orally, for imaging the stomach and small bowel although substances with specific magnetic properties may be used. Most commonly, a paramagnetic contrast agent, usually a gadolinium compound, may be given as a contrast agent. Gadolinium-enhanced tissues and fluids appear extremely bright on T1-weighted images. This provides high sensitivity for detection of vascular tissues, e.g. tumors, and permits assessment of brain perfusion, e.g. in relation to stroke.

When administered, the contrast agent finds its way through the bloodstream until it reaches the tissue of interest, such as the breast tissue, for the first time. It then takes some time, such as 6-10 minutes, to enhance the breast tissue. The enhancement is observed for some time by acquiring subsequent images using MRI. Typically, a time series of stacks of images or image volumes are acquired, starting before the enhancement and continuing for 8-10 minutes. In some cases, a maximum intensity, e.g. a peak occurring approximately 2 minutes after the start of the first image data acquisition, may be observed, which maximum is correlated with malignancy according to Kuhl C K, Mielcareck P, Klaschik S, Leutner C, Wardelmann E, Gieseke J, Schild H H, Dynamic Breast MR Imaging: Are Signal Intensity Time Course Data Useful for Differential Diagnosis of Enhancing Lesions? Radiology, 1999; 211:101-110, hereinafter referred to as Kuhl 1999. The enhancement of the breast tissue may be observed some minute(s) before and after the peak.

In some cases no peak may be observed. The tissue continuous to enhance throughout the image acquisition or the enhancement becomes approximately constant and a plateau is established.

A MRI scan is commonly regulated by a timing scheme comprising information about how the image data should be collected temporally, i.e. over a period of time, in the MRI system. In simplification, in MRI after excitation of protons of an object with an RF pulse, a number of RF pulses or so called RF profiles returning from the object are measured during a certain time, and subsequently a Fourier Transform is used to create an image. Different profiles contribute differently to the final image, e.g. the central part of the profile space (or k-space) contains the low spatial frequencies in the image. The data information in the k-space is important in order to achieve a desired image result. From an implementation point of view, the k-space is the temporary image space in which data from digitized MRI signals are stored during data acquisition. When the k-space is full, meaning at the end of the scan, the acquired data may be mathematically processed to produce a final image.

The peak of maximum enhancement, if it happens, occurs at a certain moment. Depending on the chosen k-space ordering, the peak may or may not coincide with the time when the center of the k-space is acquired. This center of the k-space contains the signal to noise and contrast information for the image and as such it contributes largely to the acquired image. So, the peak may or may not be visible in the image, however a visible peak is naturally desired.

The theory regarding the peak of maximum enhancement occurring approximately 2 minutes after bolus injection may be replaced by e.g. pharmacokinetic modeling that optionally may lead to more knowledge on the enhancing tissue. The present invention according to some embodiments may be extended to also include such pharmacokinetic modeling in order for the acquisition to be optimized for the model.

The arrival time of the contrast bolus may be determined by an injection protocol comprising information regarding injection speed and quantity of contrast agent, and information of the blood flow that differs from patient to patient, especially for patients with cardiac problems.

Throughout this specification the term “dynamic scan” refers to a MRI time series of image stacks or image volumes. One stack of images or one image volume, as part of such a time series, is referred to as a “dynamic image dataset”.

In a dynamic MRI scan having a certain duration, e.g. 80 s/dynamic image dataset, i.e. when one volume of image data is acquired every 80 seconds, image data may be acquired differently in time, depending on how k-space is sampled.

The data in k-space may be acquired in various different orders, which is indicated in FIGS. 1a to 1c. FIG. 1a illustrates a Linear Space Encoding order in which data is acquired along straight lines working from one side to the other of the k-space. FIG. 1b illustrates a Centric Space Encoding order in which data is acquired along straight lines starting in the middle of the k-space working outwards. FIG. 1c illustrates a Radial Phase Encoding order in which data is acquired along straight lines originating from a point in the centre of the k-space. Depending on the choice of timing scheme the acquired image data should be analyzed differently.

Presently, the entire scanning sequence including timing scheme comprising the start of dynamic scan, bolus injection, start of later dynamic image datasets is calculated by hand. An estimate is made, usually two minutes, as to how much time the bolus takes to arrive at the breast, and how long does the tissue take to enhance. Based on this time estimate, a second dynamic image dataset may be acquired two minutes after the bolus has started. The injector and image scanner are programmed manually with the calculated timing schemes. The manual calculation is a cumbersome procedure.

Moreover, a further problem with the current manual approach of calculating the timing scheme is related to the fact that the analysis software, such as CAD software, makes assumptions about the scanning protocol used. Usually these assumptions are implicit and many users are not aware of these. The scanning protocol and the obscure parameters involved may influence the MRI image dataset resulting from subsequent image analysis of the acquired image data.

Accordingly, current injector timing schemes are based on a certain manually calculated scanning protocol. If one chooses a different protocol, the assumed scanning protocol may be incorrect, potentially leading to misclassification of the curve type as e.g. plateau in stead of peak and accordingly to misdiagnosis.

Hence, an improved planning device, graphical user interface, and use would be advantageous, allowing for increased flexibility, cost-effectiveness, and reduced time consumption.

### SUMMARY OF THE INVENTION

Accordingly, the present invention preferably seeks to mitigate, alleviate or eliminate one or more of the above-identified deficiencies or disadvantages in the art, singly or in any combination, and solves at least the above-mentioned problems by providing a planning device, a graphical user interface, and use of the planning device according to the appended patent claims.

According to one aspect of the invention a device for planning the timing of a dynamic contrast-enhanced Magnetic Resonance Imaging scan is provided. The device is configured to receive a user-defined parameter. Moreover, the device is configured to calculate a timing scheme for said dynamic contrast-enhanced Magnetic Resonance Imaging scan at least based on said user-defined parameter.

According to another aspect of the invention a graphical user interface for planning the timing of a dynamic contrast-enhanced Magnetic Resonance Imaging scan is provided. The graphical user interface is configured to receive a user-defined parameter. Moreover, the graphical user interface is configured to calculate a timing scheme based on said user-defined parameter. Furthermore, the graphical user interface is configured to present said timing scheme to a user, e.g. on a display.

According to yet another aspect of the invention, a use of the device for the calculation of the timing scheme for dynamic Magnetic Resonance Imaging of breast tissue for locating tumors is provided.

According to some embodiments, a planning device is provided and configured to calculate the timing scheme of a dynamic MRI scan, to be integrated in either the MRI scanner or a breast analysis or CAD software package. The planned scan may be transferred to the scanner by hand or by means of an ExamCard. The planning may also be used as the input for an analysis or CAD software package.

The planning device according to some embodiments is easy to use, and it will facilitate the user in producing a proper scan and injection protocol in an error-free way.

Moreover, the timing scheme calculated by the planning device according to some embodiments may subsequently be forwarded to an analysis or CAD package.

When in communication with an external device, such as a scanner, the planning device may be configured to control a scan and injection protocol tailored to the individual patient, e.g. in the case of known cardiac problems. In this way several injection devices may be controlled remotely, e.g. by the scanner.

### BRIEF DESCRIPTION OF THE DRAWINGS

These and other aspects, features and advantages the invention is capable of will be apparent and elucidated from the following description of embodiments of the present invention, reference being made to the accompanying drawings, in which

FIGS. 1a to 1c illustrate three ways of acquiring data in the k-space. FIG. 1a illustrates a Linear Space Encoding order to acquire k-space data, FIG. 1b a Centric Space Encoding order to acquire k-space data, and FIG. 1c a Radial Phase Encoding order to acquire k-space data;

FIG. 2 is an illustration of a graphical user interface according to an embodiment;

FIG. 3 is a diagram showing a simulation of various MRI scans;

FIG. 4 is an illustration of a graphical user interface according to an embodiment; and

FIG. 5 is an illustration showing a device according to an embodiment.

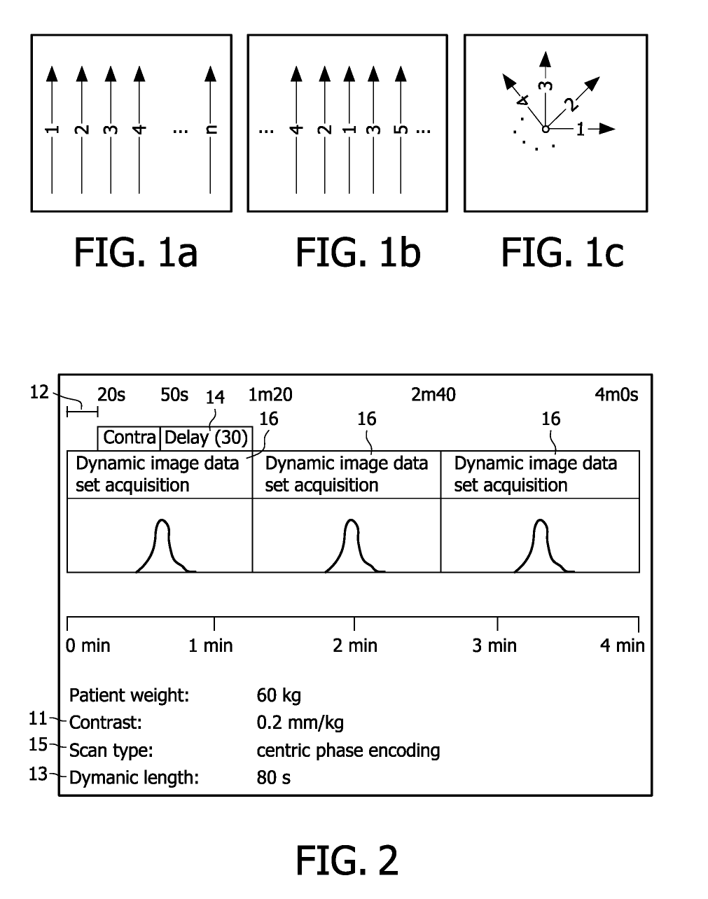

### DESCRIPTION OF EMBODIMENTS

Several embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting the invention.

The following description focuses on embodiments of the present invention applicable to dynamic MRI scan and in particular to a planning tool for calculation of a timing scheme for dynamic MRI scan of breast tissue. However, it will be appreciated that the invention is not limited to this application but may be applied to other tissue types or organs, such as prostate etc., potentially comprising tumors.

The present invention according to some embodiments is of importance for dynamic contrast-enhanced MRI of the breast, and it may be generalized to any MR dynamic scan.

According to an embodiment, a planning device comprising computer software for use in conjunction with a Breast MRI Analysis or CAD system is provided. The planning device is configured to calculate the timing scheme of an entire sequence of image scanning and injection of a contrast agent. The planning device may comprise a graphical user interface for illustrating the sequence of events as a function of time based on the timing scheme. FIG. 2 illustrates an example of a graphical user interface representation of the planning device. The graphical user interface may comprise a window illustrating a number of dynamic image datasets 16 to be acquired of an organ or tissue of a patient. Throughout each dynamic scan, one or more dynamic image datasets may be acquired. The planning device allows a user to input a number of parameters. The user-defined parameters may affect parameters of the injection protocol, e.g. injection speed, contrast agent concentration 11, total amount of contrast agent, and injection delay 12, i.e. time after start of acquiring the first dynamic image dataset of the number of dynamic image datasets before the injection is performed. The user-defined parameters may also affect parameters of the scanning protocol, e.g. dynamic image dataset acquisition duration 13, any delays, such as time between injection and acquisition of the number of dynamic image datasets in the dynamic scan 16, hereinafter called acquisition delay 14, phase encoding order 15, etc. The phase encoding order is the order in which the signal is sampled in the k-space. The most common solution is to acquire the signal along lines in one direction. Various ways of acquiring data are possible, for example, working from left to right or from the centre outwards, or having the lines evenly distributed.

The planning device may be further configured to calculate at least a parameter of the injection protocol or scanning protocol based on at least one inputted user-defined parameter. For example, if one would like to derive the timing based on the given phase encoding order 15 and certain assumptions about cardiac function, and the user has knowledge e.g. that the heart of a patient pumps less blood per second than the average patient, this user-defined parameter may be inputted into the planning device and an acquisition delay, pertaining to the term “a parameter” above, may be calculated by the planning device.

The planning device may also be configured to present a calculated parameter in a display, such as in the graphical user interface of the planning device in FIG. 2.

### Timing Scheme Calculation

The timing scheme of a dynamic contrast-enhanced MRI scan of the breast is critical for any subsequent analysis or CAD. It is also complicated to compute. Parameters having impact on the timing scheme are e.g. the bolus size or amount of the contrast agent, timing relative to the dynamics, such as flow or targeting dynamics, of the contrast agent, the delay needed for the contrast agent to travel through the venous and arterial system to the target region, the start of the pre- and post-contrast dynamic image datasets acquisition, and the phase encoding order of the MRI scan protocol.

In an embodiment at least one calculated parameter pertains to the timing scheme of the scanning procedure.

In a practical implementation of providing a timing scheme, at least three parameters are necessary: the time to acquire one dynamic image dataset, k-space ordering, and cardiac flow. The time to acquire one dynamic image dataset may be calculated from a number of scanner parameters including, e.g. field of view, resolution, number of slices, slice thickness, as well as various parameters specific to the MR pulse sequence. Instead of redoing the calculation that is to be performed by the scanner, the planning tool according to some embodiments is configured to receive the parameter pertaining to the time to acquire one dynamic image dataset. In another embodiment the planning device is configured to retrieve the parameter from a previous, similar scan via the DICOM header.

The k-space ordering, such as a centric space encoding order, pertains to the parts of the dynamic scan that have the strongest impact on the resulting dynamic image datasets.

The time from injection to the moment when the contrast agent arrives at the breast may be estimated, e.g. either by assuming a standard time like 30 s, or by modeling this process using patient mass and cardiac function. Similarly, the occurrence of the peak in the second acquisition may be assumed to take place after 120 s.

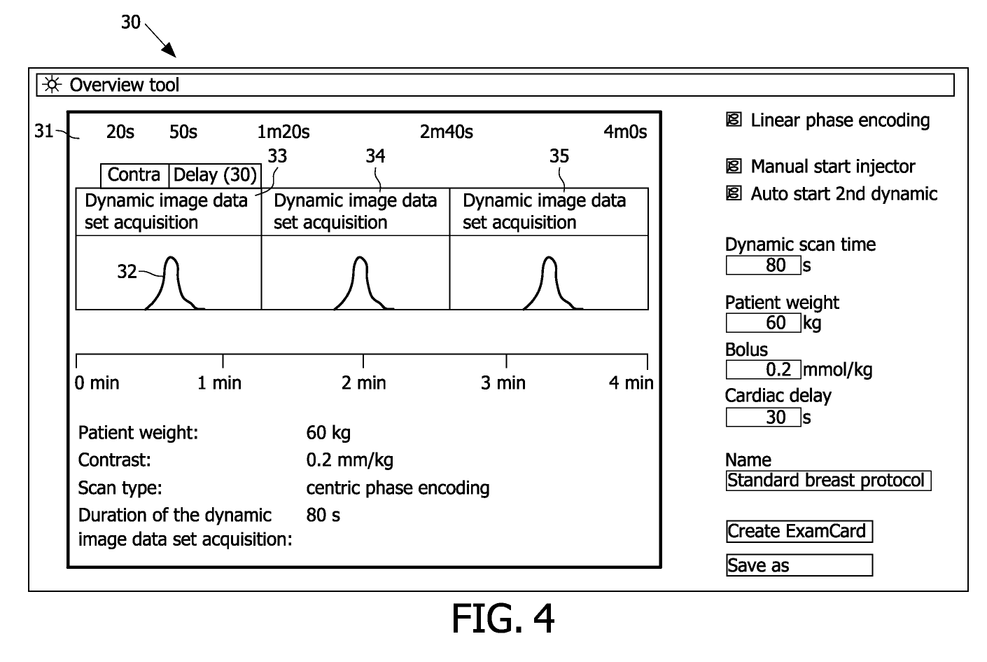

For example, if the user does not want any contrast in the first dynamic image dataset, then a point in time is established before which no contrast agent may be administered. This is illustrated in FIG. 2 or FIG. 4 as 30 s before the middle of the first dynamic image dataset.

Moreover, should the user want the second dynamic image dataset to coincide with the 2-minute peak, then this requirement teaches when to start injecting the bolus. This is illustrated in FIG. 2 or FIG. 4 as 120 s before the middle of the dynamic image dataset acquisition.

Should there be a conflict between the two timings, the planning device is configured to present the problem to the user, e.g. by presentation of problem-related information in a display such as is illustrated in FIG. 2. This conflict could also imply that a delay e.g. suggested by the device, is needed between the start of the contrast bolus injection and the acquisition of the second dynamic image dataset. This provides an advantage over current manually performed methods, wherein the user has to check any potential conflict by hand.

Table 1 illustrates how different parameters influence the timing scheme of a dynamic MRI scan.

TABLE 1

| | |
|---|---|
| Parameter | Parameter influence |

Injection protocol parameters:

| | |
|---|---|
| Injection speed | When the injection speed is higher, the |
| | same bolus is delivered in a shorter time, |
| | i.e. an earlier peak due to more condensed |
| | or non-blurred bolus may be observed. |
| | Should be modeled. |
| Bolus concentration | When the bolus concentration is higher, |
| | more contrast agent is delivered, i.e. an |
| | earlier peak due to more condensed or non- |
| | blurred bolus may be observed. |
| | Should be modeled. |
| | When it is too high, it causes clipped signals |
| | in arteries. |
| | (Note that in an MRI scan the signal intensity |
| | is not a linear function of the contrast |
| | concentration. Instead, above certain |
| | concentrations the signal intensity no longer |
| | increases and may even decrease. This is |
| | typically observed in blood vessels, where |
| | the concentration is highest.) |
| Bolus amount | Derived from patient weight |
| | (e.g. 0.1 mmol/kg). |
| Injection delay | Changing this parameter will cause the peak |
| | to appear at a different time, or not at all. For |
| | example, 20 s in FIG. 2. |

Scanning protocol parameters:

| | |
|---|---|
| Image dataset | Changing this parameter will cause the peak |
| acquisition time | to appear at a different time, or not at all. |
| Acquisition delay | Changing this parameter will cause the peak |
| | to appear at a different time, or not at all. For |
| | example, 30 sec in FIG. 2. |
| Phase encoding order | Will cause the main information in a dynamic |
| | image dataset to correspond to the middle of |
| | the acquisition of the dynamic image dataset, |
| | or the beginning, or end, or the entire dynamic |
| | image dataset acquisition time. |

Patient specific parameters:

| | |
|---|---|
| Patient Weight | Determines contrast bolus size. |
| Blood pumping rate | Peak will occur later when patient has low |
| | cardiac function. |

### Calculated Parameters

In an embodiment the planning device may be configured to enable a calculated parameter to be used in subsequent image analysis of the acquired image data of the scanning. The enablement may be accomplished by storing a calculated parameter in a memory from which an external device or a system implemented by computer software means may retrieve the calculated parameter. In this way, not only is the timing scheme for planning the examination calculated and presented, but also the information about the timing scheme may be used for subsequent image analysis.

For practical purposes, in current solutions only the scanner has the information regarding how long a certain scan with certain parameters has been or should be.

In an embodiment, when the planning device is connected to a scanner, it is configured to retrieve information regarding the dynamic image dataset acquisition time from the scanner.

In another embodiment, when the planning device is not connected to a scanner but instead, e.g. is connected to another system, such as a PACS system, dedicated medical workstation or other, information about the dynamic image dataset acquisition time may not be retrieved. In this case the planning device is configured to estimate the dynamic image dataset acquisition time, e.g. utilizing information about a previously performed dynamic scan.

In an embodiment the calculation of a parameter may be further based on previously calculated parameters, i.e. parameters calculated during previous scans, and the user-defined parameters. Accordingly, the timing scheme for the scan may be derived from a previous scan or from information available in public or private DICOM attributes.

### ExamCard

In an embodiment the planning device is configured to generate an ExamCard containing information regarding the entire scan. An ExamCard is a complete description of an examination consisting of multiple scans, including timing, etc. This embodiment is especially advantageous when the planning device is connected to or is comprised in the scanner, as the ExamCard in this case may not need to be transferred to another device.

However, in the event that the planning device is not always connected to the scanner the ExamCard could be transferred using any memory means, such as, e.g., memory stick, to the scanner.

### Scan Simulation

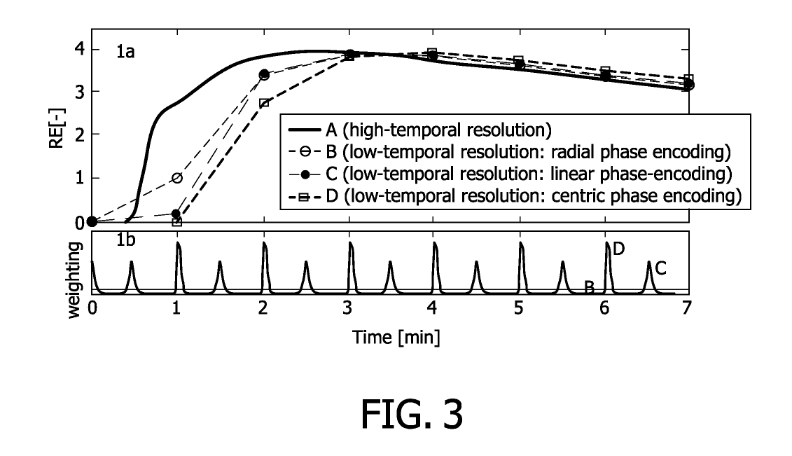

In an embodiment the planning device is further configured to simulate the entire scan, making assumptions about cardiac flow and tissue type. A contrast agent is administered intravenously, and has to pass the heart and arteries to arrive at breast tissue. The effect of this passage on the bolus as a function of time may be modeled as a delay and a blur of the bolus. If cardiac flow is less than average, meaning that the patient has a heart condition, the delay and blur become longer. If vascularity in the breast tissue is reduced, the blur increases. An example of the output of such a simulation of various scans is shown in FIG. 3. This may facilitate assessment of whether the timing scheme calculated by the planning device is acceptable.

### Controlling an External Device

Moreover, the planning device may be further configured to control an external device, such as an injector and/or MRI system, based on the user-defined and/or calculated parameters. The simplest interface between a scanner and an injection device is a panel on the scanner screen being connected to the planning device for alerting a user when to inject the bolus. The next step is to have the scanner arranged for sending a trigger signal to bolus injection. More elaborate interfaces, wherein the scanner is arranged to control the contrast bolus injection process parameters, e.g. the bolus flow (volume/second) as a function of time, could also be used, resulting in more advantages.

### Optimization of the Procedure

In some embodiments the planning device is configured to optimize the procedure. In a simple implementation the user inputs all parameters. Hence, no optimization is needed.

In a practical example of the above mentioned “non optimization case” the user sets e.g. the dynamic image dataset acquisition time to 90 s using linear phase encoding. The user also inputs the timing of the contrast bolus injection, e.g. at the beginning of the scan. The planning device is then configured to calculate when the first injection, second injection, etc., of the contrast bolus is to be expected in the breast tissue. Moreover, the planning device may be configured to show the result to the user in relation to when the dynamic image datasets are acquired having strongest contribution in the middle of each acquisition of a dynamic image dataset. This allows the user to understand his acquisition and correct it, if necessary.

However, in another example, in the event that the user would like to have a peak N seconds after bolus arrival e.g. at the breast tissue, and inputs only a few scan parameters, the planning device may be configured to calculate the remaining required scan parameters, e.g. contrast bolus start time and contrast bolus delay to obtain an optimal peak.

Alternatively, the user may just input “I want to have the center of the acquisition of the first dynamic image dataset to coincide with the first passage of the contrast bolus through the breast tissue”, and the planning device may then calculate the injection time relative to the scanning sequence.

FIG. 4 illustrates an example of the graphical user interface according to the invention, for use in connection with the planning device. The window 31 shows an overview of various events in time. The “gauss-like” curve 32 corresponds to linear phase encoding, where the centre of k-space is acquired in the middle of the dynamic image dataset acquisition 33. Similarly, the centre of k-space is acquired in the middle of the dynamic image dataset acquisition 34 and 35. In this example, that happens 1 minute 10 seconds after the end of the injection of contrast agent. The text below the graphs in window 31 displays the main parameters of this protocol such that an overview of all that is clinically relevant is provided. This information may be printed, e.g. for inclusion in a protocol handbook, and/or in ExamCard help information.

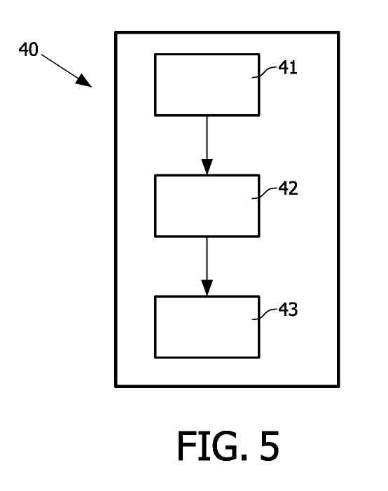

FIG. 5 illustrates a device 40 suitable for planning the timing scheme of a dynamic contrast-enhanced Magnetic Resonance Imaging scan. The device 40 is configured to receive 41 a user-defined parameter. The device may also be configured to calculate 42 a timing scheme for said dynamic contrast-enhanced Magnetic Resonance Imaging scan at least based on said user-defined parameter.

According to an embodiment the calculation of a timing scheme is performed by calculating 43 at least a further parameter based on said user-defined parameter, and said further parameter is required for calculating said timing scheme.

Applications and use of the above-described embodiments according to the invention are various and include all fields in which dynamic MRI scanning is used.

In an embodiment the planning device is comprised in a medical workstation or medical system, such as a Magnetic Resonance Imaging (MRI) System.

The planning device according to some embodiments comprises computer software for performing the above-identified functions and features. In an embodiment the computer software may reside on a computer-readable medium.

The invention may be implemented in any suitable form including hardware, software, firmware or any combination of these. However, preferably, the invention is implemented as computer software running on one or more data processors and/or digital signal processors. The elements and components of an embodiment of the invention may be physically, functionally and logically implemented in any suitable way. Indeed, the functionality may be implemented in a single unit, in a plurality of units or as part of other functional units. As such, the invention may be implemented in a single unit, or may be physically and functionally distributed between different units and processors.

Although the present invention has been described above with reference to the described embodiments, it is not intended to be limited to the described embodiments. Rather, the invention is limited only by the accompanying claims. Embodiments other than those described above are equally possible within the scope of the appended claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A device (40) for planning timing of a dynamic contrast-enhanced Magnetic Resonance Imaging scan, said device being configured to:
receive (41) a user-defined parameter; and
calculate (42) a timing scheme for said dynamic contrast-enhanced Magnetic Resonance Imaging scan at least based on said user-defined parameter.

2. The device according to claim 1, wherein said calculation of said timing scheme is performed by calculating (43) a further parameter based on said user-defined parameter, and wherein said further parameter is required by said timing scheme.

3. The device according to claim 1, wherein the user-defined parameter comprises information regarding:
an injection protocol comprising injection speed, contrast agent concentration (11), total amount of contrast agent, or injection delay;
a scanning protocol comprising duration of an acquisition of the dynamic image dataset, acquisition delay, or sampling mode of k-space in time; or
a patient characteristic comprising patient's weight or blood flow.

4. The device according to claim 2, wherein said further calculated parameter pertains to an injection protocol or scanning protocol.

5. The device according to claim 1, being comprised in an MRI analysis package, a CAD system, or an MRI scanner.

6. The device according to claim 5, wherein said timing scheme is utilized in subsequent image analysis.

7. The device according to claim 1, further configured to generate an ExamCard.

8. The device according to claim 1, further configured to control an injector.

9. The device according to claim 1, wherein the calculation of the timing scheme can use a parameter from a previously performed dynamic scan or a previously calculated timing scheme.

10. The device according to claim 1, further configured to visualize (43) said timing scheme on a display.

11. The device according to claim 1, further configured to simulate a dynamic scan based on said timing scheme.

12. The device according to claim 1, wherein said timing scheme comprises duration of an acquisition of a dynamic image dataset comprised in the dynamic scan, k-space ordering or cardiac flow.

13. The device according to claim 1, further configured to retrieve a parameter for the timing scheme from an external device.

14. The device according to claim 1, wherein said user-defined parameter comprises information regarding user preferences.

15. The device according to claim 14, configured to display user-defined parameters conflicting with each other in the calculated timing scheme.

16. A graphical user interface (30) for planning the timing of a dynamic contrast-enhanced Magnetic Resonance Imaging scan, said graphical user interface being configured to
receive a user-defined parameter,
calculate a timing scheme based on said user-defined parameter, and
present said timing scheme to a user.

17. Use of the device according to claim 1 for the calculation of the timing scheme for a dynamic Magnetic Resonance Imaging scan of breast tissue for locating tumors.
